(19) United States
(12) Patent Application Publication
(10) Pub. No.: US 2025/0118421 A1
(43) Pub. Date: Apr. 10, 2025

(54) Artificial Intelligence Medical Coding System

(71) Applicants: Sean Boutros, M.D., F.A.C.S., Houston, TX (US); Roberto Secchi, Houston, TX (US); Sathvik Kakanuru, Redmond, WA (US)

(72) Inventors: Sean Boutros, M.D., F.A.C.S., Houston, TX (US); Roberto Secchi, Houston, TX (US); Sathvik Kakanuru, Redmond, WA (US)

(21) Appl. No.: 18/907,115

(22) Filed: Oct. 4, 2024

Related U.S. Application Data
(60) Provisional application No. 63/542,386, filed on Oct. 4, 2023.

(51) Int. Cl. G16H 40/20

(52) U.S. Cl. CPC G16H 40/20

(57) ABSTRACT

A system and method for determining a medical code corresponding to a medical procedure description using artificial intelligence algorithms includes a Current Procedural Terminology (CPT) code set that includes records each including a code associated with a medical condition description, respectively. The system includes a deep learning neural network in communication with said CPT code set that is trained using medical data that is updated in real-time using at least medical literature obtained using natural language processing (NLP). The method includes using NLP to receive a medical procedure description and comparing the NLP-enhanced description to the code set until a match is made. That match represents the proper code associated with the NLP-enhanced description.

### REFERENCE TO RELATED APPLICATIONS

This application claims the benefit of provisional patent application No. 63/542,386 filed Oct. 4, 2023, which was titled Conversion of Medical Descriptions to Standardized|Billing Codes Using Artificial Intelligence Algorithms and which is incorporated herein in its entirety.

### BACKGROUND OF THE INVENTION

This invention relates generally to healthcare technology and, more particularly, to medical coding and billing using artificial intelligence (AI) algorithms. It is intended to automate and streamline the process of converting procedure labels to Current Procedural Terminology (CPT) codes and match them with the respective ICD-10 code which is used for medical billing and insurance claims.

Traditionally, hospitals and medical groups include one or more employees referred to as “coders” who are tasked with taking medical records in which a physician or other medical professional has described a medical procedure and, upon review, assign an appropriate medical billing code by which insurance companies will then be consistently and correctly billed for payment. There may be lengthy and complicated billing code standards that are intended to correctly match medical procedure descriptions with corresponding billing codes. Needless to say, this overall clerical procedure can become very complicated, be overwhelmed with inconsistent billing, and experience numerous errors that must be later reviewed again and changed by another layer of insurance company coders. In other words, the insurance company may employ numerous coders whose primary purpose may be to disagree with the medical group or hospital coders and to change the billing codes.

The clinic or hospital coder may then submit a claim to a respective insurance company where the diagnosis and code are once again reviewed—this time by an “insurance coder department which is composed of coders, nurses and physician.” If the code is still incorrect, the claim will be denied. The substitute diagnosis is given by the insurance company and the diagnosis/diagnoses are paid. Once the clinic or hospital are made aware of this, their claims department writes an appeal or dispute to substantiate the original diagnostic code(s). The clinic or hospital appeals department documents criteria that they believe will allow the original diagnostic code(s) to be reimbursed.

Various medical billing systems have been proposed throughout the years. Although presumably effective for their intended purposes, a common characteristic of such systems is that they still include multiple layers of claim audits, coding changes, and a lack of feedback to the treating provider. The proposed invention is a novel combination of AI technologies and medical coding that is non-obvious to those skilled in the field due to the complexity and specialty knowledge required in both medical coding and AI model training.

Therefore, it would be desirable to have a system and method for medical coding and billing that uses artificial intelligence algorithms to automate and accurately convert medical procedure descriptions to corresponding medical billing codes according to the CPT protocol A major challenge of implementing the present system was ensuring that the AI algorithms accurately understood and interpreted the medical procedure labels in context. This was overcome by extensive training on the 2023 CPT code set, as well as on the ICD-10 code list and implementing a feedback loop for continuous learning and improvement.

### SUMMARY OF THE INVENTION

Accordingly, a computer implemented system for determining a medical code corresponding to a medical procedure description using at least one deep learning algorithm of artificial intelligence includes a non-transitory computer readable storage medium containing program code and data structures. The system includes a current procedural terminology (CPT) code stored in the storage medium that includes a plurality of records each including a code associated with a respective medical procedure description, respectfully. The CPT code set is in communication with a deep learning neural network that is trained using medical data that is updated in real time using at least medical literature obtained using natural language processing (NLP).

The system may include a processor in data communication with the storage medium and that is operative to execute program code configured to perform method steps including receiving input data indicative of a description of a medical procedure, applying natural language processing (NLP) to the input data so as to generate a filtered description of the medical procedure description, and comparing the filtered description to the plurality of records in the code set until a matching record is located. The matching record will include the associated code and which may be published to a digital display were saved in a corresponding data structure.

Stated another way, The invention solves the problem of time-consuming and error-prone manual CPT, ICD-10 coding. It achieves this through automated coding and validation, providing a more accurate and efficient process compared to existing methods. ChatGPT and LongChain algorithms process and interpret the procedure descriptions, map them to appropriate CPT codes, as well as match the appropriate disease described with the respective ICD-10 code and check the operation note for accuracy and formatting. These components interact to convert descriptions into accurate CPT codes, facilitating an efficient billing process. The feedback loop is used through multiple iterations to improve the accuracy with which the model is able to convert to the codes.

Essential components include the AI models (ChatGPT and LongChain) and the training on tCe 2023 CPT and ICD-10 code set, as these are fundamental for the interpretation and mapping of procedure descriptions.

The Chatgpt-4.0-turbo model is being used to guarantee high levels of accuracy and short runtimes. In the future, with the evolution of AI, this may be modified, but at the present time, this is the only large language model capable of the level of reasoning required to achieve this goal for the current budget.

Therefore, a general object of this invention is to provide a system and method for medical coding and billing that uses artificial intelligence algorithms to automate and accurately convert medical procedure descriptions to corresponding medical billing codes according to the CPT protocol.

Other objects and advantages of the present invention will become apparent from the following description taken in connection with the accompanying drawings, wherein is set forth by way of illustration and example, embodiments of this invention.

### BRIEF DESCRIPTION OF THE DRAWINGS

FIG. 1 is a perspective view of a method for determining a medical code from a medical procedure description according to the present invention;

FIG. 2 is a block diagram illustrating a system according to the methodology as in FIG. 1, illustrated by showing a computing device in data communication with the Internet for accessing and determining a CPT Code set and the like;

FIG. 3 is a block diagram illustrating that the system for determining a medical code includes the computing device; and

FIG. 4 is a flowchart illustrating elements of artificial intelligence that are utilized to accomplish the method of determining a medical code from a medical procedure description according to the present invention.

### DESCRIPTION OF THE PREFERRED EMBODIMENT

A medical coding and billing system using artificial intelligence according to a preferred embodiment of the present invention will now be described with reference to the accompanying drawings.

In a preferred embodiment, the computer implemented system 10 for determining a medical code corresponding to a medical procedure description may include a computing device 100 such as a smart phone, a laptop computer, a digital tablet, a computer workstation, or the like. Preferably, the computing device 100 includes a processor 102 in data communication with a non-transitory storage medium 104 (also referred to as a memory), the processor 102 being configured to execute program steps such as computer programming 105 that may be stored in the memory 104. The memory 104 may also include data structures 105a which are embodied as memory locations capable of storing data. It will be understood that the referenced programming and data structures may be included in the form of a mobile application 110 which may also be referred to as computer software. The computing device 100 may include a digital display 108 on which a medical procedure or corresponding code may be published during or after identification as will be described below in greater detail. Preferably, the mobile application 110 will include various aspects of artificial intelligence algorithms many of which will be described in greater detail later. Preferably, the computer implemented system 10 is in data communication with other digital resources via a wide area network such as the Internet 150.

In another critical aspect, the system 10 includes a current procedural terminology (CPT) code set 20 that is stored in the storage medium 104 that includes a plurality of records each including a code associated with a respective medical procedure description, respectfully. The CPT code set 20 may be in communication with a deep learning neural network that is trained using medical data that is updated in real time using at least medical literature obtained using natural language processing (NLP).

Current Procedural Terminology (CPT) codes are a uniform system of medical codes that are used to describe medical procedures and services. The American Medical Association (AMA) creates and maintains CPT codes, which are used for a variety of purposes, including:
Billing: CPT codes are used for billing and insurance coverage and payment.
Data analysis: CPT codes are used for data analysis.
Communication: CPT codes provide a uniform language for communication between physicians, patients, and third parties.
Administrative purposes: CPT codes are used for administrative purposes such as claims processing and developing guidelines for medical care review.

Traditionally, CPT codes are five digits long, and each code represents a distinct procedure or service. The AMA regularly updates CPT codes to reflect current clinical practice and innovation in medicine.

There are multiple ways to get updates 22 on the Current Procedural Terminology (CPT) code set 20, including accessing the following digital resources, such as via the Internet 150.
CPT News: A free monthly email newsletter from the American Medical Association (AMA) that provides updates on the CPT code set and related industry news.
AMA website: The AMA website regularly posts updates on CPT codes.
CPT & RBRVS Annual Symposium: The AMA hosts an annual symposium that provides guidance on CPT code updates.
AMA Ed Hub™: The AMA Ed Hub™ provides guidance on CPT code updates.
CPT Network: The CPT Network provides guidance on CPT code updates.
Healthie: Healthie is an EHR and practice management solution that provides real-time updates on CPT code changes.
AMA Storefront on Amazon: The AMA Storefront on Amazon sells coding books and products, including the CPT Professional Edition codebook.
AMA Intelligent Platform: The AMA Intelligent Platform provides CPT data products, including the CPT Standard Data File.

The AMA releases new editions of the CPT code set four months before the January 1 operational date. The CPT Editorial Panel, an independent body convened by the AMA, manages changes to the CPT code set.

In a related aspect, medical procedure labels are used in healthcare settings to help ensure patient safety, improve communication, and prevent errors. Some types of medical procedure labels include:
Pre-surgical labels, also known as surgical work labels or pre-operative labels, these labels help organize and document information and tasks related to surgical procedures.
Laboratory labels, these labels help identify patients and streamline the lab testing process.
Anesthesia labels, these labels communicate the type and amount of anesthesia being used during surgery.
Blood bag labels, these labels help medical professionals determine which blood bag to use for each patient.
Auxiliary labels, also called cautionary and advisory labels or prescription drug warning labels, these labels are added to a dispensed medication package by a pharmacist.

Further, medical device labels may include the following information:
Device identification, including name, description, and intended use
Manufacturer identification, including name and full address
Lot number or serial number
Unique Device Identifier (UDI)

The American Medical Association (AMA) offers several ways to download current CPT code sets, including:
AMA Store, the AMA Store offers print and digital versions of the codebook, data products, and online coding subscriptions. The AMA Store also sells coding and reimbursement e-books.
AMA Intelligent Platform, the AMA Intelligent Platform offers CPT data products, including the CPT Standard Data File. The 2024 Standard Data File includes six descriptor types, section guidelines, and appendices.
CPT Smart App, the CPT Smart App is a digital platform that allows users to request modifications or additions to the CPT code set.

The AMA also offers a vaccine code finder resource to help identify the appropriate CPT code for COVID-19 vaccines and administration services.
List of CPT/HCPCS Codes—CMS
   The American Medical Association (AMA) provides even more description of medical labels and updates for the CPT codes and which can be found at https://www.ama.assn.org/practice-management/cpt/cpt-overview-and-code-approval#:˜:text:Designated%20by%20the%20U.S.%20Department,developing%20technology%2C%20procedures%20and%20services.

In another critical aspect, the present system 10 may include program code that implements the artificial intelligence 200 concept of Natural language processing (NLP) 202, which is a subfield of computer science and artificial intelligence (AI) in which uses machine learning to enable computers to understand and communicate with human language.

The following website https://www.ibm.com/topics/natural-language-processing#:˜:text=Natural%20language%20processing%20(NLP)%20is,and%20communicate %20with%20human%20language describes operation of natural language processing as follows:
Natural language processing (NLP) is a subfield of computer science and artificial intelligence (AI) that uses machine learning to enable computers to understand and communicate with human language.
NLP enables computers and digital devices to recognize, understand and generate text and speech by combining computational linguistics—the rule-based modeling of human language-together with statistical modeling, machine learning (ML) and deep learning.
NLP combines the power of computational linguistics together with machine learning algorithms and deep learning. Computational linguistics is a discipline of linguistics that uses data science to analyze language and speech. It includes two main types of analysis: syntactical analysis and semantical analysis. Syntactical analysis determines the meaning of a word, phrase or sentence by parsing the syntax of the words and applying preprogrammed rules of grammar. Semantical analysis uses the syntactic output to draw meaning from the words and interpret their meaning within the sentence structure.
The parsing of words can take one of two forms. Dependency parsing looks at the relationships between words, such as identifying nouns and verbs, while constituency parsing then builds a parse tree (or syntax tree): a rooted and ordered representation of the syntactic structure of the sentence or string of words. The resulting parse trees underly the functions of language translators and speech recognition. Ideally, this analysis makes the output-either text or speech-understandable to both NLP models and people.
Self-supervised learning (SSL) in particular is useful for supporting NLP because NLP requires large amounts of labeled data to train state-of-the-art artificial intelligence (AI) models. Because these labeled datasets require time-consuming annotation-a process involving manual labeling by humans-gathering sufficient data can be prohibitively difficult. Self-supervised approaches can be more time-effective and cost-effective, as they replace some or all manually labeled training data.

In another aspect, a fallback loop 204 may be utilized in the artificial intelligence programming to enhance the accuracy of natural language processing. The fallback interaction is triggered when your chatbot doesn't recognize the user's message. A fallback loop is configured to ask the user to reword their question, show answers they can choose from or transfer them to a human agent.

In a related aspect, LongChain 206 is an open-source framework that helps developers create applications that use natural language processing (NLP). It's built around Large Language Models (LLMs) 208, which are trained on large amounts of human language to understand and generate language. LongChain uses a combination of components to help applications process and respond to human language, including large language models which is another aspect of artificial intelligence. For example, the core of LongChain, LLMs are trained on large datasets to generate coherent and relevant text.

LongChain 206 can be used to develop a variety of applications, including chatbots, virtual agents, intelligent search, question-answering, and summarization services. Some of its advantages include streamlining the development of AI application interfaces, improving access to and processing of large amounts of data, overcoming limitations in the knowledge base of language models.

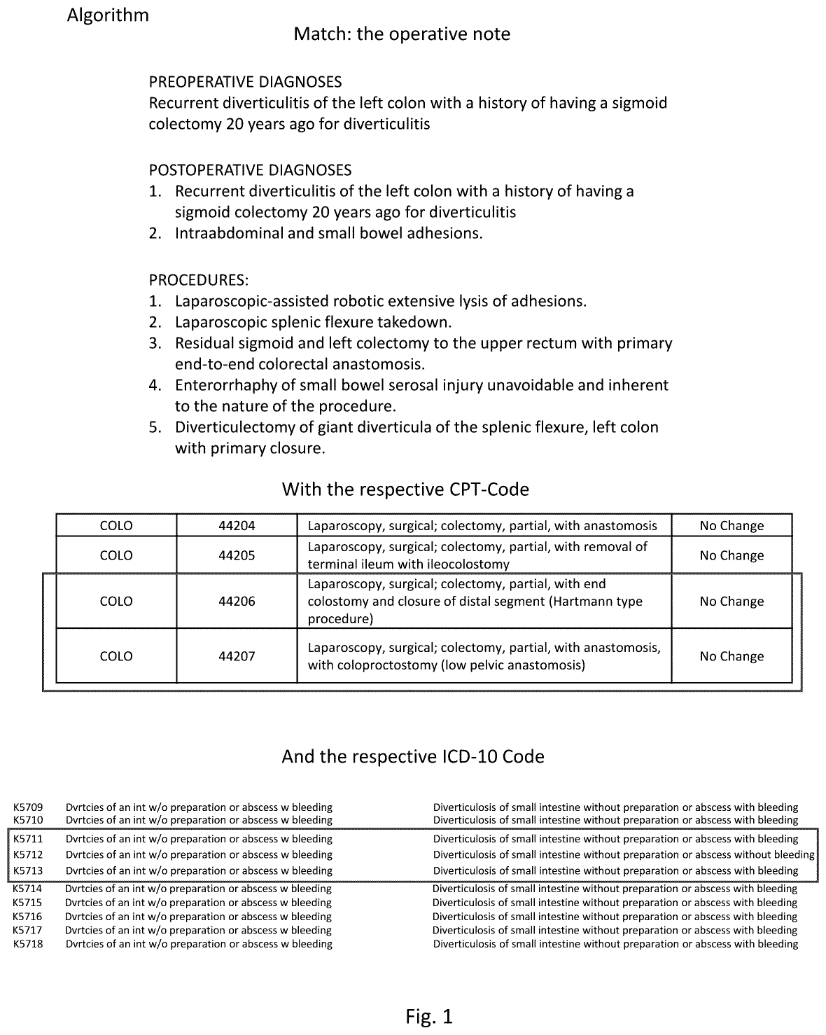

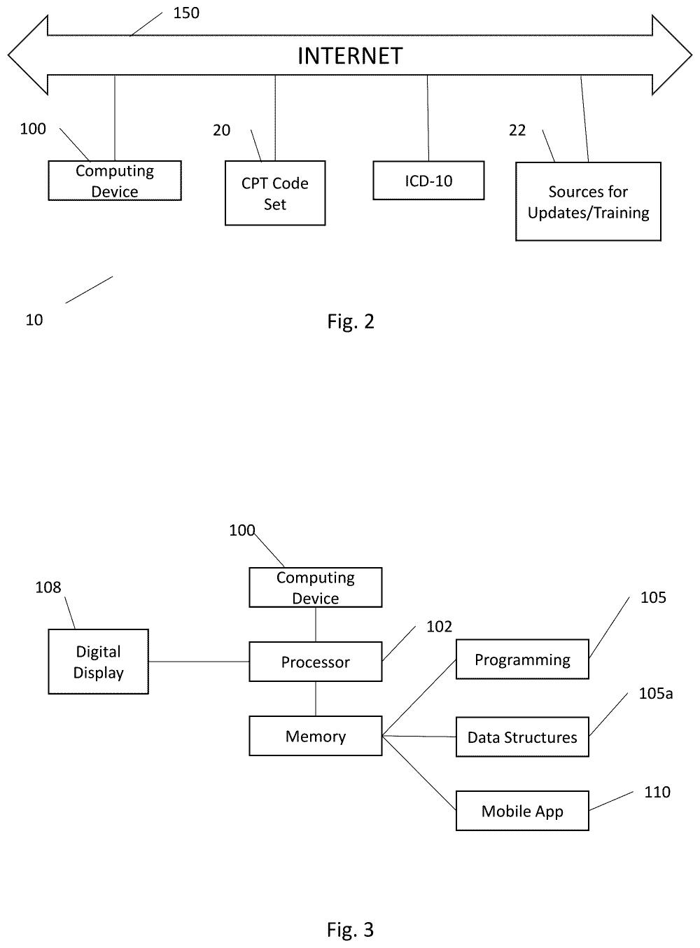

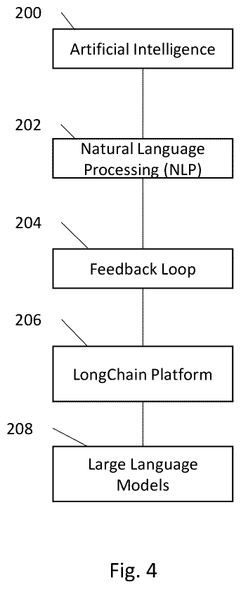

The present invention includes method steps that are analogous to the system 10 described in the pages above. For instance, the method according to the present invention includes automating the very complicated methodology of recognizing a medical procedure description and then selecting a CPT or ICD-10 numerical code, as shown in FIG. 1. Get the other figures, of course, illustrate the system components and their data communication via the Internet 150 described previously.

It is understood that while certain forms of this invention have been illustrated and described, it is not limited thereto except insofar as such limitations are included in the following claims and allowable functional equivalents thereof.

What is claimed is:

1. A computer implemented system for determining a medical coding corresponding to a medical procedure description using at least one deep learning algorithm of artificial intelligence, said system comprising:
a non-transitory computer-readable storage medium containing program code and data structures;
a Current Procedural Terminology (CPT) code set stored in said storage medium, said CPT code including a plurality of records each including a code associated with a medical condition description, respectively;
a deep learning neural network in communication with said CPT code set that is trained using medical data that is updated in real-time using at least medical literature obtained using natural language processing (NLP);
a processor in data communication with said computer-readable storage medium that is operative to execute said program code to perform the steps of:
receiving input data indicative of a description of a medical procedure;
applying natural language processing (NLP) to said input data so as to generate a filtered description of the medical procedure; and procedure description
comparing said filtered description of the medical procedure to said plurality of records in said CPT code set until a matching record is located.

2. The system as in claim 1, wherein said processor is operative to execute said program code to perform the step of publishing said matching record to a digital display.

3. The system as in claim 1, wherein said deep learning neural network is trained using data obtained in real-time from extensive training using the most up-to-date CPT code set.

4. The system as in claim 1, wherein said deep learning neural network is trained using data obtained in real-time from extensive training on the most up-to-date ICD-10 code list.

5. The system as in claim 1, wherein the extensive training of said deep learning neural network includes implementing a feedback loop that is configured to request human clarification or amending of a respective medical procedure description if, at first, the NLP did not find a respective matching record.

6. The system as in claim 1, wherein said NLP is configured to recognize, understand, and generate text and speech associated with a respective medical description using computational linguistics along with statistical modeling, large language models (LLMs), machine learning (ML) and deep learning techniques.

7. The system as in claim 6, wherein said NLP is part of a LongChain framework that is configured to determine accurate CPT codes after being trained on large language models.

8. A method for determining a medical code corresponding to a medical procedure description using at least one deep learning algorithm of artificial intelligence, said method comprising:
a Current Procedural Terminology (CPT) code set, said CPT code including a plurality of records each including a code associated with a medical condition description, respectively;
a deep learning neural network in communication with said CPT code set that is trained using medical data that is updated in real-time using at least medical literature obtained using natural language processing (NLP);
receiving input data indicative of a description of a medical procedure;
applying natural language processing (NLP) to said input data so as to generate a filtered description of the medical procedure; and procedure description; and
comparing said filtered description of the medical procedure to said plurality of records in said CPT code set until a matching record is located.

9. The method as in claim 8, further comprising publishing said matching record to a digital display.

10. The method as in claim 8, wherein said deep learning neural network is trained using data obtained in real-time from extensive training using the most up-to-date CPT code set.

11. The method as in claim 10, wherein said deep learning neural network is trained using data obtained in real-time from extensive training on the most up-to-date ICD-code list.

12. The method as in claim 8, wherein said NLP is configured to recognize, understand, and generate text and speech associated with a respective medical description using computational linguistics along with statistical modeling, large language models (LLMs), machine learning (ML) and deep learning techniques.

13. The method as in claim 8, wherein said NLP includes a LongChain framework that is configured to determine accurate CPT codes after being trained on large language models.
